(19) United States
(12) Patent Application Publication
(10) Pub. No.: US 2007/0073156 A1
(43) Pub. Date: Mar. 29, 2007

(54) Combined visual-optic and passive infra-red technologies and the corresponding systems for detection and identification of skin cancer precursors, nevi and tumors for early diagnosis

(75) Inventors: Arkadii Zilberman, Beer Sheba (IL); Yafim Smolyak, Kiriat Ono (IL); Nathan Blaunshtein, Beer Sheba (IL); Ben Zion Dekel, Hadera (IL); Avraham Yarkony, Beer Sheba (IL)

Correspondence Address: DR. MARK M. FRIEDMAN;C/O BILL POLKINGHORN - DISCOVERY DISPATCH, 9003 FLORIN WAY, UPPER MARLBORO, MD, 20772, US

(73) Assignees: Yafim Smoliak, Kiriat Ono (IL); Passive Imaging Medical Systems Engineering Ltd., Tel Aviv (IL)

(21) Appl. No.: 11/464,838

(22) Filed: Aug. 16, 2006

Related U.S. Application Data
(60) Provisional application No. 60/708,389, filed on Aug. 16, 2005.

(51) Int. Cl. A61B 6/00

(52) U.S. Cl. 600/473

(57) ABSTRACT

A device and method to non-invasively identify pathological skin lesions. The method and device detect and identify of different kinds of skin nevi, tumors, lesions and cancers (namely, melanoma) by combined analyses of visible and infra-red optical signals based on integral and spectral regimes for detection and imaging leading earlier warning and treatment of potentially dangerous conditions.

This is a continuation-in-part of U.S. Provisional Patent Application No. 60\708389, filed Aug. 16, 2005.

### FIELD AND BACKGROUND OF THE INVENTION

The present invention relates to a non-invasive method and device to identify pathological skin lesions. More specifically the present invention relates to a method and device for non-intrusive detection and identification of different kinds of skin nevi, tumors, lesions and cancers (namely, melanoma) by combined analyses of visible and infra-red optical signals based on integral and spectral regimes for detection and imaging leading earlier warning and treatment of potentially dangerous conditions.

Commonly suspicious lesions are biopsied to determine their status. Biopsies have many obvious disadvantages: firstly biopsies require intrusive removal of tissue that can be painful and expensive. Only a very limited number of sights can be biopsied in one session and patients are not likely to put up with a large number of such expensive painful tests. Furthermore, biopsy samples must be stored and transported to a laboratory for expert analysis. Storage and transportation increase the cost, increases the possibility that samples will be mishandled, destroyed or lost, and also causes a significant time delay in receiving results. This time delay means that examination follow up requires bringing the patient back to the doctor for a separate session. This increases the inconvenience to the patient, the cost and the risk that contact will be lost or the disease will precede to a point of being untreatable. Furthermore, the waiting period causes significant anxiety to the patient. Finally, interpretation of biopsies is usually by microscopic analysis, which results in qualitative subjective results, which are not well suited to consistent interpretation.

Therefore, in medical diagnosis there is great interest in safe, non-intrusive detection technologies, particularly, in the case of skin cancer. Cancer is a disease that develops slowly and can be prevented by monitoring Lesions with potential to become cancerous through routine screening. There is, nevertheless, a limit to the amount of time, money or inconvenience that a basically healthy patient is willing to dedicate to routine screening procedures. Therefore, screening must be able to reliably identify dangerous tumors and differentiate dangerous tumors for benign nevi (moles) quickly, inexpensively and safely.

There are many methods for spectral analysis and imaging of skin anomalies using active regimes, which are widely known. These methods have used not only optical spectral and thermal imaging methods, visible and infrared, but also electromagnetic microwave, acoustic, magnetic, ultraviolet and X-ray methods [see for example Fear, E. C., and M. A. Stuchly, “Microwave detection of breast tumors: comparison of skin subtraction algoritluns”, SPIE, vol. 4129, 2000, pp. 207-217; Gniadecka, M., “Potential for high-frequency ultrasonography, nuclear magnetic resonance, and Raman spectroscopy for skin studies”, Skin Research and Technology, vol. 3, No. 3, 1997; and Bruch, R., et al, “Development of X-ray and extreme ultraviolet (EUV) optical devices for diagnostics and instrumentation for various surface applications”, Suface and Interface Anal. vol 27, 1999, pp. 236-246].

X-ray technology, which has been used successfully for detection of anomalies inside the human-body since the early 60's, is not suited for earlier detection of skin cancer because, due to it's the dangerous effects of X-ray radiation on human health, it cannot be used often enough (weekly or monthly), for diagnostics of patients with skin anomalies which need intensive reexamination over short-time periods.

Acoustic active methodologies, which are useful for detection of structures inside the human body, are also non-effective for early diagnosis cancerous skin anomalies. Precancerous skin lesions are often of microscopic dimensions (on the order of millimeters or micrometers), which cannot be detected and identified by use acoustic methods (which are limited to detecting structures larger than the wavelength of sound on the order of centimeters).

Microwave detection of skin tumors, nevi or cancer is based on the contrast in dielectric properties of normal and anomaly skin tissues. Microwave technologies are very complicated and radiate the human body with microwave radiation, which may have dangerous effects. Furthermore, microwave signals with wavelength from few mm to few cm, cannot identify small structures with diameter of half mm or less, but anomalies on the half mm scale are very important in early cancer diagnosis [Bruch, R., et al, “Development of X-ray and extreme ultraviolet (EUV) optical devices for diagnostics and instrumentation for various surface applications”, Surface and Interface Anal. vol. 27, 1999, pp. 236-246].

Optical methods for detection, identification and diagnosis of skin abnormalities have been applied in order to avoid the above disadvantages of tradition biopsies and their interpretation. Optical methods can be classified into two regimes. The first is called the integral regime of skin structure detection. In the integral regime infrared the spatial distribution of a signal is measured to obtain information about changes in skin properties (like temperature of color), which mark the boundaries between normal skin and anomalous regions. The second regime is called the spectral regime. In the spectral regime radiation intensities are measured in various frequency bands generally based on reflected light in the visible to NIR bands. The spectral regime is useful for identification of specific anomalies based on information about the corresponding “signature” of the anomaly in the frequency domain.

There are many methods for spectral analysis and imaging of skin lesions. Generally the analysis uses an active regime, applying radiation from an external source and measuring the reflection, absorption and refraction of the rays. These non-intrusive methods reduce cost and lead to objective quantitative results. Furthermore, when physical sampling is necessary, samples, for spectral analysis, may be smaller than traditional biopsies. This makes the sampling procedure significantly less traumatic for the patient. Spectral analyzers may even be brought to a doctor's office or an operating room to allow real time diagnosis and treatment considerably increasing the efficiency of treatment as well as reducing expensive and dangerous time delays and reducing the chance of losing contact with patients. Nevertheless, all of the widely known techniques such as optical imaging, optical spectral analysis, and thermal imaging have disadvantages making them not fully appropriate for detection and identification of skin cancer and cancer precursors.

One optical spectroscopy technique for non-invasive detection of skin cancer proposed by BC Cancer Research Centre includes analysis of absorption and scattering properties of the skin in visual waveband (400-750 nm) and autofluorescence spectra of the skin. Chemical and structural changes due to skin diseases lead to characteristic autofluorescence and diffuse reflectance spectra. These spectral features can be use to differentiate skin cancer from other skin diseases. Using reflectance spectra alone, it would be difficult to differentiate between various skin conditions since different skin diseases have similar reflectance spectra. By considering the corresponding fluorescence spectrum for a particular skin disease, it is often possible to differentiate between skin anomalies that have similar reflectance spectra. Nevertheless, being a purely spectral method limited to the visible frequency band, this method does not give important information about the geometry of a lesion. Also some lesions can be difficult to identify positively even with both fluorescence and reflectance spectra. For example the fluorescence intensity of a Seborrheic kertosis may be higher or lower than normal skin depending on the lesion thickness and degree of hyperkeratosis. Therefore it would be desirable to have further identifying information on a lesion to positively identify the lesion, its stage of development and the danger to the patient.

Another optical system for identifying skin lesions is MelaFind, which was created by Electro-Optical Sciences Inc. (EOS) to non-invasively detect early melanoma. The principle of operation is based on multispectral image analysis (multispectral dermoscopic images are used as the input for subsequent computer analysis). Diagnostic process includes: step 1—Multispectral imaging; Step 2—Segmentation (Removing hairs, segmenting lesion); and Step 3—Extracting and analyzing features. A probe uses reflected light to image the lesion. Ten images are obtained using different narrow-spectrum wavelengths from the NIR through visible light spectrum to obtain information on the absorption and scattering properties of the lesion. This provides information about the lesion border, size, and morphology that is not available to the naked eye. A specialized imaging probe detects illumination in each spectral band, creates the digital images and sends them to computer for processing. The methodology lacks the ability to make a full spectral analysis in real time and therefore positively identify the color and shade of the lesion and is therefore not able to positively differentiate all kinds of benign, percancerous and cancerous lesions. The method does not give precise information on the depth of the lesion.

Another optical method is based on a device known as a DermLite. The method uses cross-polarized no-oil epiluminescence microscopy for improved diagnosis of pigmented skin lesions and basal cell carcinoma. The DermLite incorporates cross-polarization filters that reduce reflection of light from the surface of the skin and permits visualization of the deeper structures. Light from white Light Emitting Diodes (LEDs), is polarized linearly by a special filter and the image viewed through a magnifying lens is also linearly polarized so as to cancel out the reflected light from the surface of the skin. This mode is called Cross Polarized ELM and has been extensively studied for the imaging of pigmented lesions for the early detection of melanoma. While this method allows full visible spectrum imaging of near surface lesions, it does not allow determination of the depth of the lesion. Furthermore based on a visible reflectance spectrography alone it is not possible to differentiate many pathological lesions from normal skin or nevi. For example, in FIG. 2 the difference between aggressive precancerous structures 1b and a benign nevus is only apparent due to increased absorbance in the NIR region.

Narrow band IR spectrum methodologies for analyzing and classifying skin pathologies include Raman spectroscopy [Barry, B. W., H. G. M. Edwards, and A. C. Williams, “Fourier transform Raman and infrared vibrational study of human skin: assignment of spectral bands”, Journal of Raman Spectroscopy, vol. 23, 1992, pp. 641-645; Gniadecka, M., H. C. Wulf, and N. N. Mortensen, “Diagnosis of basal cell carcinoma by Raman spectroscopy”, Journal of Raman Spectroscopy, vol. 28, 1997; Fendel, S, and Schrader, “Investigation of skin and skin lesions by NIR-FT-Raman spectroscopy”, Journal of Annal. of Chemistry, vol. 5, 1998; Sterenborg, H. J. C. M., M. Motamedi, F. Sahebkar, et al., “In vivo optical spectroscopy: new promising techniques for early diagnosis of skin cancer”, Skin Cancer, vol. 8, 1993, pp. 57-65] and methods based on infrared (IR) spectroscopic diagnostics (called Fourier-transform-infrared spectroscopy, FTIR) combined with fiber optic techniques (called fiber-optical evanescent wave method, FEW) [Afanasyeva, N., S. Kolyakov, V. Letokhov, et al, “Diagnostic of cancer by fiber optic evanescent wave FTIR (FEW-FTIR) spectroscopy”, SPIE, vol. 2928, 1996, pp. 154-157; Afanasyeva, N., S. Kolyakov, V. Letokhov, et al, “Noninvasive diagnostics of human tissue in vivo”, SPIE, vol. 3195, 1997, pp. 314-322; Afanasyeva, N., V. Artjushenko, S. Kolyakov, et al., “Spectral diagnostics of tumor tissues by fiber optic infrared spectroscopy method”, Reports of Academy of Science of USSR, vol. 356, 1997, pp. 118-121; Afanasyeva, N., S. Kolyakov, V. Letokliov, and V. Golovkina, “Diagnostics of cancer tissues by fiber optic evanescent wave Fourier transform IR (FEW-FTIR) spectroscopy”, SPIE, vol. 2979, 1997, pp. 478-486; Bruch, R., S. Sukuta, N. I. Afanasyeva, et al., “Fourier transform infrared evanescent wave (FTIR-FEW) spectroscopy of tissues”, SPIE, vol. 2970, 1997, pp. 408-415; Brooks, A., R. Bruch, N. Afanasyeva, et al., “Investigation of normal skin tissue using fiberoptical FTIR spectroscopy”, SPIE, vol. 3195, 1997, pp. 323-333; Afanasyeva, N., S. Kolyakov, L. N. Butvina, “Remote skin tissue diagnostics in vivo by fiber optic evanescent wave Fourier transform infrared spectroscopy”, SPIE, vol. 3257, 1998, pp. 260-266; Brooks, A., N. Afanasyeva, R. Bruch, et al., “Investigation of human skin surfaces in vivo using fiber optic evanescent wave Fourier transform infrared (FEW-FTIR) spectroscopy”, Surface and Interface Analysis, vol. 27, 1999, pp. 221-229; Brooks, A., N. Afanasyeva, R. Bruch, et al., “FEW-FTIR spectroscopy applications and computer data processing for noninvasive skin tissue diagnostics in vivo”, SPIE, vol. 3595, 1999, pp. 140-151; Sukuta, S., and R. Bruch, “Factor analysis of cancer Fourier transform evanescent wave fiber-optical (FTIR-FEW) spectra”, Lasers in Surgery and Medicine, vol. 24, No. 5, 1999, pp. 325-329; Afanasyeva, N., L. Welser, R. Bruch, et al., “Numerous applications of fiber optic evanescent wave Fourier transform infrared (FEW-FTIR) spectroscopy for subsurface structural analysis”, SPIE, vol. 3753, 1999, pp. 90-101]. These techniques use a narrow spectral waveband from 3-5 ?m or from 10-14 ?m (MIR fiber-optics spectroscopy [Artjushenko, V., A. Lerman, A. Kryukov, et al., “MIR fiber spectroscopy for minimal invasive diagnostics”, SPIE, vol. 2631, 1995]). These narrow band IR methods are effective for differentiating normal skin from abnormal tissue. Nevertheless, being limited to measurements of narrow band IR these methods cannot detect subtle differences between a non-pathologic nevus and an early cancer precursor. These methods cannot even reliably differentiate nevi from skin cancer, since as is shown in FIG. 2, nevi have their characteristic maxima in the visible optics spectrum, and cannot be positively identified using only the IR regime.

Parallel with IR spectrography, the method of thermal imaging uses optical cameras to produce color images of skin tumors or skin pathological anomalies. This passive integral regime method detects differences in patterns of IR emissions from normal and pathological tissues. The results of this imaging are generally classified into four main parameters. The parameters are then used for detection and identification of pathological and benign skin anomalies (e.g. tumors, melanoma, lesions and nevi). The parameters are: a) asymmetry of the anomaly shape; b) bordering of the anomaly; c) color of the anomaly; d) dimensions of the anomaly. The main limitations of thermal imaging are that thermal cameras are limited in their ability to detect very fine temperature differences associated with precancerous lesions and that without spectral data it is nearly impossible to positively differentiate benign and aggressive lesions based on the integral regime alone.

Hyperspectral imaging method (HIM) proposed by SIAscopy company is a passive method based on a spectral regime. HIM uses a selective spectrum range, using several narrow wavebands. Because it doesn't include a continuous spectrum, the HIM method cannot give information about shade and color features of ill and healthy tissue. Thus HIM is not very good at detecting subtle changes in precancerous lesions. Furthermore, lacking an integral component HIM does not measure the geometry and particularly the depth of a lesion.

Method of AstronClinics (MAC) company is a passive method based on the spectral regime in selective frequency bandwidths according to requirements of a dermatologist. It also includes an integral regime, which measures the gradient of temperature for imaging of structure of the skin anomaly. Measurement of temperature gradients is ineffective when the temperature of the anomaly is close to the temperature of the regular skin structure. The main disadvantage of the spectral regime of this method is that because it is limited to a few narrow frequency bands, it cannot obtain complete information about color and shade, which are basic parameters of a melanoma.

The method for imaging DIRI [Melnik B. “Optical Diagnostics of Skin Cancer,” M.Sc. Thesis, Ben-Gurion Univ. 2004] is based on integral regime of measurements of the patterns and distribution IR radiation (an IR camera is used). This method is not fully passive since it requires heating of tissue with the corresponding anomaly, such as nevi or melanoma, by IR radiation and afterwards observing the heat flow and rate of temperature decrease during cooling of a lesion. In this method gradients of temperature are also observed. A spectral regime measurement is performed selectively using only some frequencies bands from whole spectrum. The method has poor resolution and identification of the anomalies of interest because it is affected by noise and clutter. Also, because the method lacks information on depth and includes measurement only of visible band radiation, the method has low degree of identification. Another disadvantage of the method is that it requires the additional operations of heating and cooling the skin.

There is thus a widely recognized need for, and it would be highly advantageous to have, a non-invasive methodology to identify all kinds of pathologic skin conditions and particular early cancer precursors. The current invention fills this need by employing a differential measure to improve sensitivity to subtle differences in intensity of visible and infrared emission from the skin. This improved sensitivity allows precise quantification of changes in light absorption and heat generation in the skin that are characteristic of different forms of skin lesions and stages of cancer development. Therefore the present invention discloses an extremely sensitive method to differentiate between normal skin cells and those with pathological anomalies. For example, in embodiments described below, the current invention uses the differential measure contrast between the normal skin cell and skin cells with pathological anomalies in an integral regime and a spectral regime of skin analysis. Spatial distribution of contrast of a wide frequency band is taken into account in the integral regime to detect a lesion and to assess the position, size and shape of the lesion. Frequency dependence of the contrast, its magnitude and its sign are used to assess, vascular and metabolic activity, which are different for normal skin and skin with pathological anomalies. Combined together, both regimes allow precise diagnostics different skin anomalies and facilitate earlier warning of cancerous and precancerous conditions. As a non-invasive method, the proposed invention allows researchers to use non-destructive testing of any skin anomaly.

### SUMMARY OF THE INVENTION

The present invention is a non-invasive method and device to identify pathological skin lesions. More specifically the present invention relates to a method and device for non-intrusive detection and identification of different kinds of skin nevi, tumors, lesions and cancers (namely, melanoma) by combined analyses of visible and infra-red optical signals based on integral and spectral regimes for detection and imaging leading earlier warning and treatment of potentially dangerous conditions.

According to the teachings of the present invention there is provided a non-intrusive method for identifying a lesion in a skin of a subject. The method includes the steps of measuring a radiation to find a location of an anomaly of the radiation emitted by the skin. The anomaly is caused by the lesion. Then a spectral analysis is performed by quantifying a first signal in a visual band and a second signal in an infrared band. The lesion is then identified based on the measured location and a result of the spectral analysis

According to the teachings of the present invention, there is also provided a detector for identifying a lesion in a skin. The detector includes a first sensor assembly sensitive to a first frequency band. The first sensor assembly is configured to determine a location and a characteristic of an anomaly in a first radiation signal emitted by the skin. The anomaly is caused by the lesion. The detector also includes a second sensor assembly configured to be sensitive to a second frequency band, and a processor configured to identify the lesion based on the measured location, the measured characteristic and a contrast between an unmodified radiation signal in the second frequency band emitted by the skin and a second radiation signal measured at the location of the lesion by the second sensor assembly.

According to further features in preferred embodiments of the invention described below, the step of identifying a lesion also includes recognizing a cancer precursor.

According to still further features in the described preferred embodiments, cancer precursor is recognized based on a measurement of an energy in a near infrared band.

According to still further features in the described preferred embodiments, the radiation that is measured includes a visible light reflected from the skin.

According to still further features in the described preferred embodiments, the measured radiation includes a visible light emitted by fluorescence of the skin.

According to still further features in the described preferred embodiments, the measured radiation includes a black body medium infrared band energy emitted by the skin.

According to still further features in the described preferred embodiments, the measured radiation includes energy in a broad frequency band including both infrared and visible frequencies.

According to still further features in the described preferred embodiments, the measured radiation includes energy in the near infrared frequency band scattered by the skin.

According to still further features in the described preferred embodiments, the measured radiation includes both a visible light reflected from the skin and a black body medium infrared band energy emitted by the skin.

According to still further features in the described preferred embodiments, the step of finding a lesion includes the substeps of quantifying a first energy emitted from the skin without the lesion and then measuring a second energy emitted from the location, where a lesion is to be detected. Then a differential measure is calculated between the first energy and said second energy.

According to still further features in the described preferred embodiments, the method further includes the step of classifying the lesion to a general category based on a characteristic of the measured radiation anomaly. After classifying the lesion to a general category, the spectral analysis is adapted to differentiate between objects in the general category.

According to still further features in the described preferred embodiments, the step step of adapting the spectral analysis includes choosing a frequency band for the spectral analysis. The chosen frequency band is optimal to distinguish between at least two objects in the general category.

According to still further features in the described preferred embodiments, the method further includes the step of determining the depth of the lesion.

According to still further features in the described preferred embodiments, the step step of finding the lesion and said step of determining the depth of the lesion are performed simultaneously.

According to still further features in the described preferred embodiments, the step step of determining the depth of the lesion includes the substeps measuring an infrared energy emitted by the lesion and computing a depth based on a resulting infrared measurement.

According to still further features in the described preferred embodiments, the method for identifying a lesion further includes the step of measuring a fluorescence, and the identification of the lesion is further based on the outcome of the measurement of fluorescence.

According to still further features in the described preferred embodiments, the step second signal in the spectral analysis includes an infrared energy having wavelength between 5.5 and 7.5 micrometers.

According to still further features in the described preferred embodiments, the step of performing a spectral analysis includes the substeps of measuring a first energy measured in a first frequency band emitted at the location of the anomaly, quantifying a second energy measured in a second frequency band emitted at that location, and calculating a differential measure between the first energy and the second energy.

According to still further features in the described preferred embodiments, the step the second signal in the spectral analysis includes a product of an interaction between an output of an external radiation source and the lesion, a heat flow from the lesion, a light reflected from the lesion, or a black body radiation emitted by the lesion.

According to still further features in the described preferred embodiments, the step the step of identifying the lesion includes classifying the lesion into one of many categories. The potential categories include a benign nevus, pathologic cancer precursor, and cancerous lesion.

According to further features in the described preferred embodiments, the first sensor assembly of the detector for a cancerous lesion includes an electronic sensor and the second sensor assembly includes the same electronic sensor and a band pass filter.

According to still further features in the described preferred embodiments, the detector of a cancerous lesions also includes a visible light source for producing a light beam, and the first sensor assembly is configured to detect a reflection of the light beam from the skin.

According to still further features in the described preferred embodiments, the detector of a cancerous lesion also includes an ultra-violet light source configured to induce fluorescence of the skin, and the second sensor is configured to detect the fluorescence.

According to still further features in the described preferred embodiments, the processor includes a human operator, a dedicated electronic processor, or a personal computer.

### BRIEF DESCRIPTION OF THE DRAWINGS

The invention is herein described, by way of example only, with reference to the accompanying drawings, where:

FIG. 1 is a first embodiment of a device to identify cancerous lesions according to the current invention;

FIG. 2 is a visible band spectrogram of light reflected from a nevus and various stages from benign to melanoma;

FIG. 3a is a spectrogram showing visible band fluorescent spectra from a seborrheic keratosis and normal skin;

FIG. 3b is a spectrogram showing visible band reflected spectra from a seborrheic keratosis and normal skin;

FIG. 3c is a spectrogram showing visible band fluorescent spectra from a compound nevus and normal skin;

FIG. 3d is a spectrogram showing visible band reflected spectra from a compound nevus and normal skin;

FIG. 4 is an IR contrast spectrogram of melanoma;

FIG. 5 is a flow chart illustrating a method do identify a cancerous lesion according to the current invention;

FIG. 6 is a second embodiment of a device to identify a cancerous lesion according to the current invention;

FIG. 7 is a third embodiment of a scanner to identify a cancerous lesion according to the current invention.

### DESCRIPTION OF THE PREFERRED EMBODIMENTS

The principles and operation of a non-invasive method and device to identify pathological skin lesions according to the present invention may be better understood with reference to the drawings and the accompanying description.

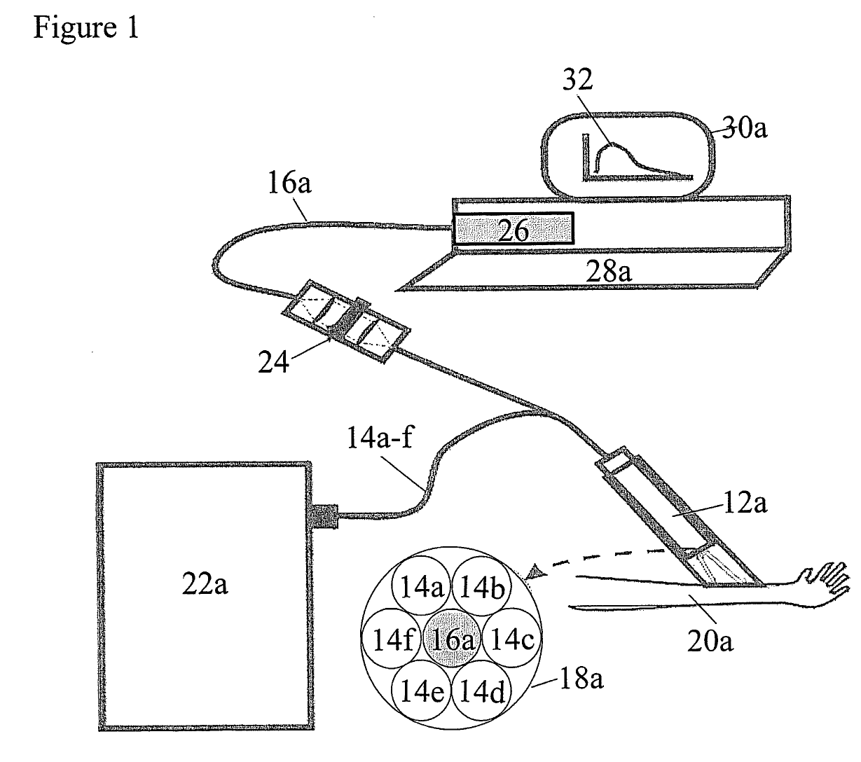

FIG. 1 illustrates a method for early detection of skin cancer according to the current invention. A skin probe 12a contains a bundle of optical fibers, including 6 illumination fibers 14a, 14b, 14c, 14d, 14e, and 14f and a pick up fiber 16a as is seen in cross sectional view 18a. Probe 12a is passed over the skin 20a of a patient. Illumination fibers 14a-f are connected to a light source 22a containing an He—Cd laser and a QTH lamp. Pick up fiber 16a is connected through an adjustable filter 24 to a spectrometer card 26, which resides in a personal computer (PC) 28a. PC 28a is provided with a monitor 30a, for display of results, for example spectrogram 32.

A wide band integral measurement in the visible frequency band is used to find the location of anomalies of reflected energy in the visible light band from skin 20a that may be a sign of pathological lesions. To make the wide band measurement, filter 24 is set to allow a wide band of light to pass through pick up fiber 16a. In the embodiment of FIG. 1 the integral measurement is made for wavelength 300-900 nm (i.e., in visual and NIR spectral bands). QTH lamp of light source 22a is activated producing a light beam in the visible and NIR bands. The light beam travels down illumination fibers 14a-f and shines on skin 20a, the light reflects off the surface of skin 20a and is transmitted along pick up cable 16a through filter 24 to spectrometer card 26. Spectrometer card 26 digitizes the signal and passes the result to PC 28a for processing. First a measurement is made of the intensity of light reflected from normal skin, the results being the overall energy flow from the regular skin structure R′. Then the area of interest of the skin is scanned to find anomalies. The resulting radiation flow measurement at the point being scanned R″ is processed by PC 28a and output as a differential measure from normal skin. In the embodiment of FIG. 1, the differential measure, contrast C is calculated according to the formula C=(R′−R″)/(R′+R″). Anomalous regions (where the absolute value of contrast is large) are identified for further investigation in the spectral regime to identify the precise status of the anomaly, whether the anomaly is a benign structure, a cancerous precursor that needs to be monitored, or a pathological lesion requiring treatment.$$$$

In the embodiment of FIG. 1 four separate measurements are made. First a measurement of a visible light signal due to fluorescence is made by using a band pass filter to set Filter 24 to allow a first narrow band ? λ₁ of visible light to pass through pick up fiber 16a and activating He—Cd laser of light source 22a to produce ultraviolet light beam. The ultraviolet light beam travels down illumination fibers 14a-f and shines on skin 20a, stimulating fluorescence in the surface of skin 20 producing a visible band light that is transmitted along pick up cable 16a through filter 24 to spectrometer card 26. Spectrometer card 26 digitizes the signal and passes the result to PC 28a for processing. PC 28a thereby measures fluorescence in a first narrow band. An operator then adjusts filter 24 to pass light in a second narrow visible band ? λ₂, and PC 28a measures fluorescence in the second band. Sequentially the user repeatedly changes filter 24 and measures the signal is a set of bands producing a fluorescence spectrum.

In the embodiment of FIG. 1, in each band ? λᵢ of the spectrum intensity R is quantified for normal skin R′?? λᵢ ? and then at a location of an anomalous region the spectrum intensity R″?? λᵢ ? is measured. The contrast, C, of spectral density of emitted radiation (dR/d??? where R is the overall radiation flow in the chosen spectral band and ? is the wavelegnth? in each spectral band, ? λᵢ, is calculated by PC 28a as follows: C(? λ₁)=[R′??λ₁−R′??λᵢ?]/[R″??λᵢ+R′??λᵢ?]

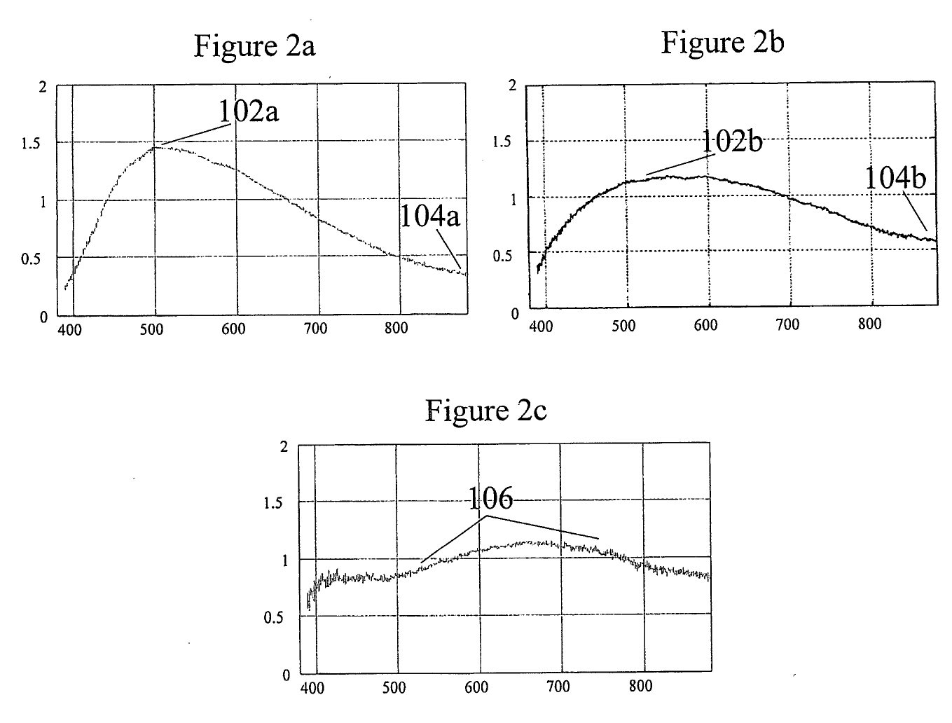

After measuring the fluorescence spectrum, the operator measures a second signal due to the reflectance of visible light by switching off the He—Cd laser and activating the QTH lamp of light source 22a. The QTH lamp produces visible light which passes through illumination fibers 14a-f shining on the surface of skin 20 and reflecting back to pick up fiber 16a. The operator the sequentially adjusts filter 24 and makes measurements with PC 28a, producing a reflected visible spectrum spectrogram (e.g. see FIG. 2) on monitor 30a.

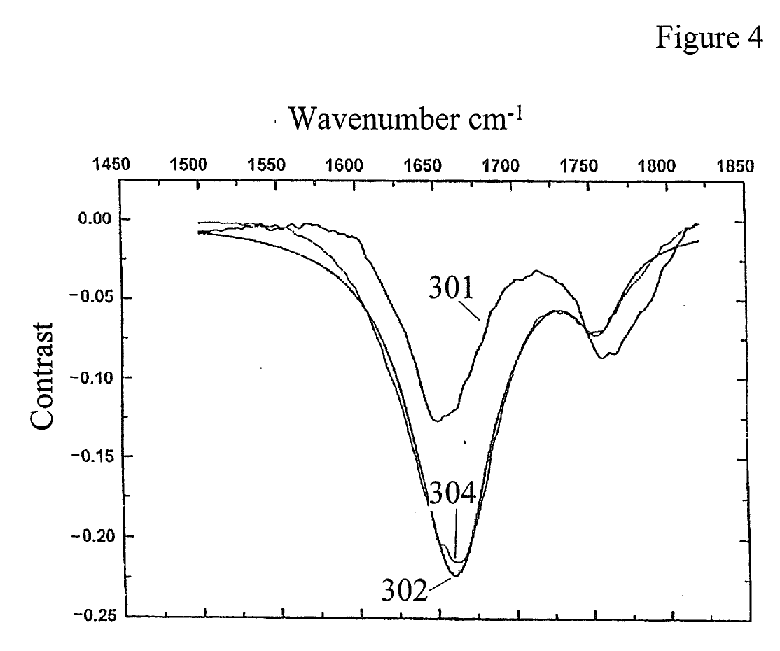

After measuring the reflected visible/NIR spectrum, the operator switches off light source 22a and adjusts filter 16a to pass light in the medium infrared (MIR) regime. Changing from band to band as described above, the operator passively measures a third signal which is a medium infrared, MIR, band spectrum (e.g. FIG. 4) from skin 20a, which is treated as a black body with temperature T₀? 36.6° C. radiating in the MIR spectral range. Thus by changing the frequency dependence of filter 24, the sensor assembly of probe 12 and spectrometer card 26 are used to measure energy in different frequency bands.

Probe 12a is also used to scan the anomalous zone in a wide band MIR (??=4−12?m) in an integral mode to outline the shape of the anomalous zone both on the surface of the skin and at depth using topographic techniques. The depth of the anomaly is most important parameter with respect to area of anomaly localization, because there is some critical depth where melanoma can be transferred in its dangerous form. Particularly, blood vessels lie a few millimeters under the skin surface, lesions that reach 7 mm depth are much more likely to metastasize and are much more dangerous than shallower lesions. Because visible light does not penetrate skin, it is difficult to determine the depth of a lesion using visible (reflectance or fluorescence) imaging.

Alternatively, the depth of a lesion can be determined using probe 12a in an active mode to measure NIR scattering. In such an embodiment, light source 22a would produce a NIR light in a narrow band around 900 nm wavelength. Such NIR light penetrates normal skin but is scattered by blood. Similarly, filter 24 is adjusted to allow NIR light to pass through pick fiber 16a. Thus, probe 12a would detect locations having increased density of blood vessels near the skin surface (a typical signal of melanoma development).

There are following experiments have been carried out to proof our invention.1) in visible frequency band:In [Melnik B. “Optical Diagnostics of Skin Cancer,” M.Sc.Thesis, Ben-Gurion Univ. 2004] were described the experiments carried out for melanoma and nevi detection and identification by use visible optics spectroscopy. About 100 mice were investigated from the initial stage of melanoma injection at the lesion, analyzing dynamic of cancer development up to the final stage of cancer evolution. Parallel, 80 patients having different kinds of nevi were observed by using this passive method. More than 60 spectrograms for different kinds of nevi were obtained. All of them showed that the normal nevus has maximum of its contrast relative to the normal lesion at 500 nm. FIG. 2a, FIG. 2b and FIG. 2c show normalized spectral characteristics of the contrast of absorbance of visible radiation by nevus obtained from a mouse during three stages of development from a nevus to a melanoma. The spectrogram of a normal nevus FIG. 2a has an obvious maximum reflectance 102a at 500 nm. Some nevi were so aggressive that after some term of several weeks they had transformed to melanoma, which has plateau shaped spectral distribution (FIG. 2c). The spectrogram of an aggressive precancerous nevus FIG. 2b, has a peak 102b at 500 nm similar to a normal nevus, but is recognized by elevated reflectance 104b in the NIR band (900 nm) in comparison to a normal nevus, which has very low reflectivity in the NIR band 104a. A developed melanoma has a plateau shaped visible reflectance spectrogram 106 as shown in FIG. 2c.

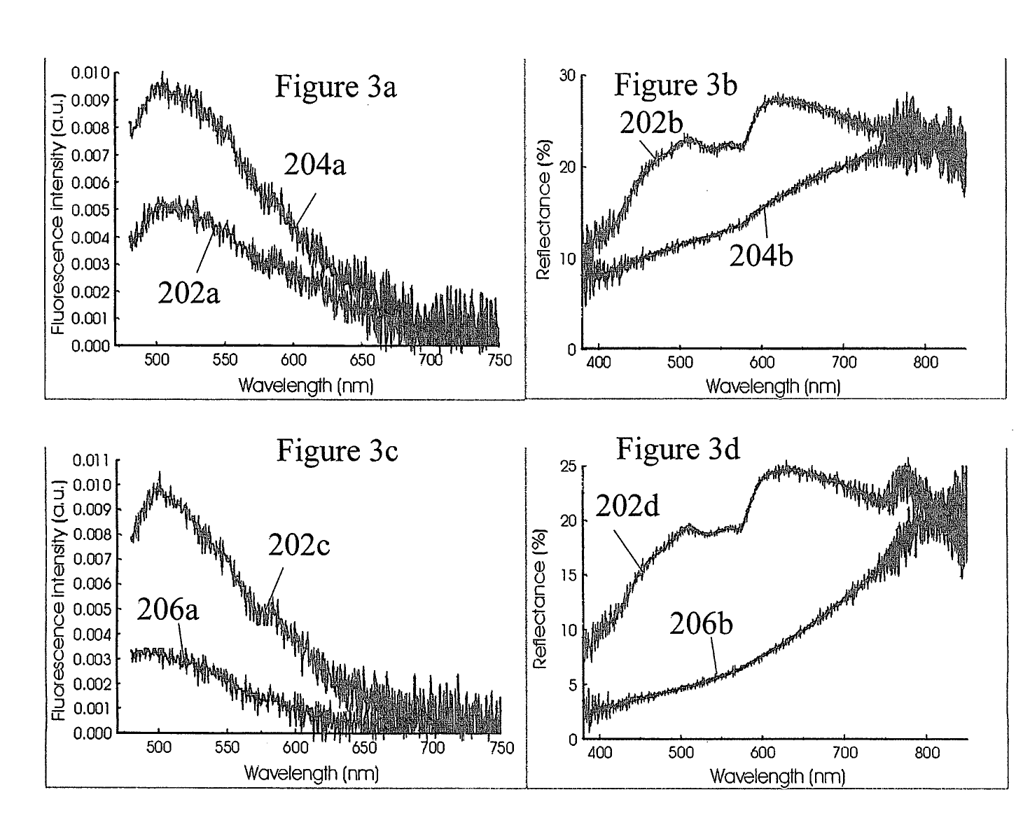

FIG. 3a and FIG. 3b show an example of typical autofluorescence FIG. 3a and diffuse reflectance spectra FIG. 3b of normal skin 202a,b and a seborrheic keratosis 204a,b. FIG. 3c and FIG. 3d show an example of typical autofluorescence FIG. 3c and diffuse reflectance spectra FIG. 3d of normal skin 202c,d and a seborrheic keratosis 206a,b. Using reflectance spectra 202b,d 204b, 206b alone or visual inspection under white light illumination, it could be difficult to differentiate between the seborrheic keratosis 204b and compound nevus 206b. However, when also considering the corresponding fluorescence spectrum for the particular skin disease, it is possible to differentiate between seborrheic keratosis 204a with a fluorescence intensity higher than normal skin and compound nevus 206a with fluorescence intensity much lower than normal skin, Nevertheless, in some cases Seborrheic keratoses can have lower fluorescence intensities than their surrounding normal skin, depending on lesion thickness and degree of hyperkeratosis.

Thus, visible light reflectance is not enough to identify many lesions (e.g. compound nevus and Seborrheic keratoses). Analyzing visible fluorescence allows identification of some of these lesions (e.g. a Seborrheic keratoses having fluorescence intensity higher than normal skin) but in some cases both (e.g. a compound nevus and a Seborrheic keratoses having fluorescence intensity lower than normal skin) there needs to be extra information. In some cases, it may not be possible to differentiate between a melanoma and a benign nevus using only the visible spectrum. In the embodiment of FIG. 1, these difficult cases are identified using IR spectroscopy.

In one alternative embodiment of the current invention, not all spectral measurements are made every location of an anomaly of the integral radiation scan. Rather, depending on a characteristic of the integral scan, the anomaly is classified into a general category and then the spectral scanning method is adapted to differentiate between specific lesions in the general category. For example, if a lesions shows increased reflectance 104b in an initial integral scan in the NIR band, then the lesion is classified as either a melanoma FIG. 2c, a precancerous compound nevus FIG. 2b, or a benign Seborrheic keratosis 204b. To differentiate these lesions, first a visible fluorescence scan is made at a 500 nm wavelength, which is the optimal wavelength to differentiate a keratosis from a compound nevis as can be seen by comparing spectrogram 204a to spectrogram 206a. If the fluorescence is elevated in relation to normal skin 204a then lesion is identified as a Seborrheic keratoses. If the fluorescence is not elevated, then a full visible reflectance spectrum is measured. If there is a maximum reflectance at 500 nm then the lesion is identified as a precancerous nevus FIG. 2b. If the visible reflectance spectrogram has a passive MIR scan is made. If the heat flow is elevated near the skin surface, then the lesion is identified as a potential shallow melanoma. If the heat flow is elevated also at depth then the lesions is identified as a potentially deep melanoma and if the heat flow is

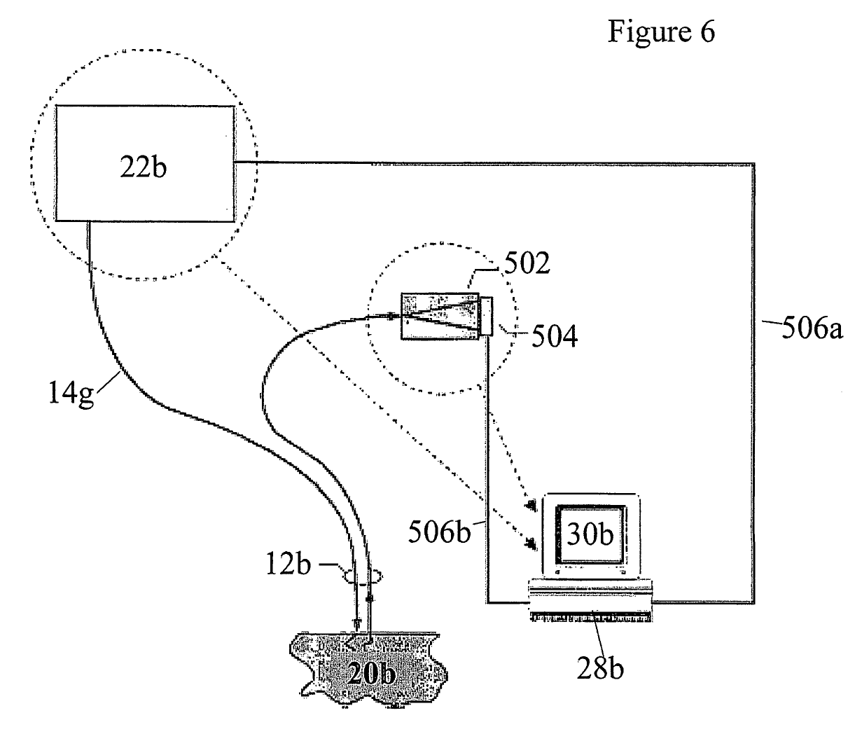

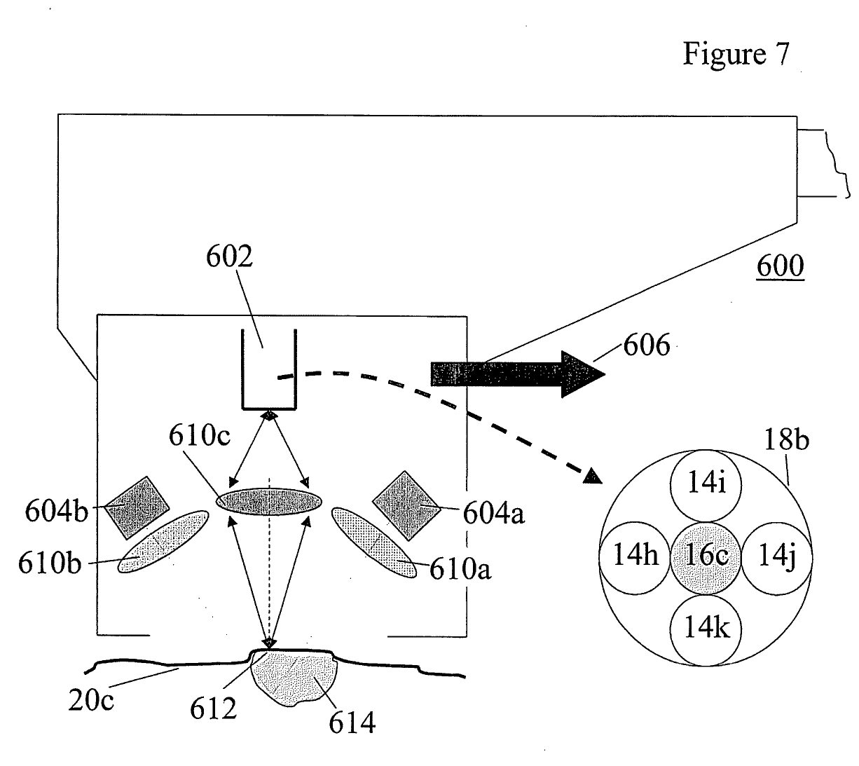

FIG. 4 illustrates three passive infrared contrast spectrograms of two types of melanoma: a measured passive IR spectrogram of a female melanoma 301 and a maile melanoma calculated theoretically 302 and measured 340. Because the measured parameter is contrast, for normal skin the spectrogram is a horizontal line at zero. Similarly, benign nevi have heat flow similar to normal skin and therefore a flat contrast of zero. It is seen that melanoma can be identified by a clear peak in the MIR band between 5-7 ?m. In fact melanoma and associated increased circulation causes a local temperature rise of the order of 0.1 K. This temperature rise results in a small increase in black body radiation from the skin. The small magnitude of this increase may not be apparent in heat imaging or to a FLIR (forward looking infrared) camera. Nevertheless, using a pyroelectric detector (for example the detector of the embodiment of FIG. 1 and FIG. 4 was acquired from ORIEL Instrument Inc, USA [also see details of measurement techniques in Brooks, A., N. Afanasyeva, R. Bruch, et al, “Investigation of human skin surfaces in vivo using fiber optic evanescent wave Fourier transform infrared (FEW-FTIR) spectroscopy”, Surface and Interface Analysis, vol. 27, 1999, pp. 221-229; Brooks, A., N. Afanasyeva, R. Bruch, et al., “FEW-FTIR spectroscopy applications and computer data processing for noninvasive skin tissue diagnostics in vivo”, SPIE, vol. 3595, 1999, pp. 140-151; Sukuta, S., and R. Bruch, “Factor analysis of cancer Fourier transform evanescent wave fiber-optical (FTIR-FEW) spectra”, Lasers in Surgery and Medicine, vol. 24, No. 5, 1999, pp. 325-329; and Afanasyeva, N., L Welser, R. Bruch, et al., “Numerous applications of fiber optic evanescent wave Fourier transform infrared (FEW-FTIR) spectroscopy for subsurface structural analysis”, SPIE, vol. 3753, 1999, pp 90-101] and processing the signal using a differential measure of IR radiation intensity (for example, in the embodiment of FIG. 1 and FIG. 4 the differential parameter contrast), this small increase is easily detected even for lesions as deep as a few centimeters under the skin surface. In the embodiment of FIG. 1 the IR spectrum is measured by sequential narrow band IR measurements using diffraction filters (as described above for measurements of visual band spectra). In alternative embodiments (see FIG. 6 and FIG. 7) simultaneous measurements are made of different narrow band signals (using multiple detectors and multiple refraction grating filters) or a single measurement is used and PC 28b computes the spectrum using Fourier transforms as in FTIR from an interferogram or other know measurement technique.

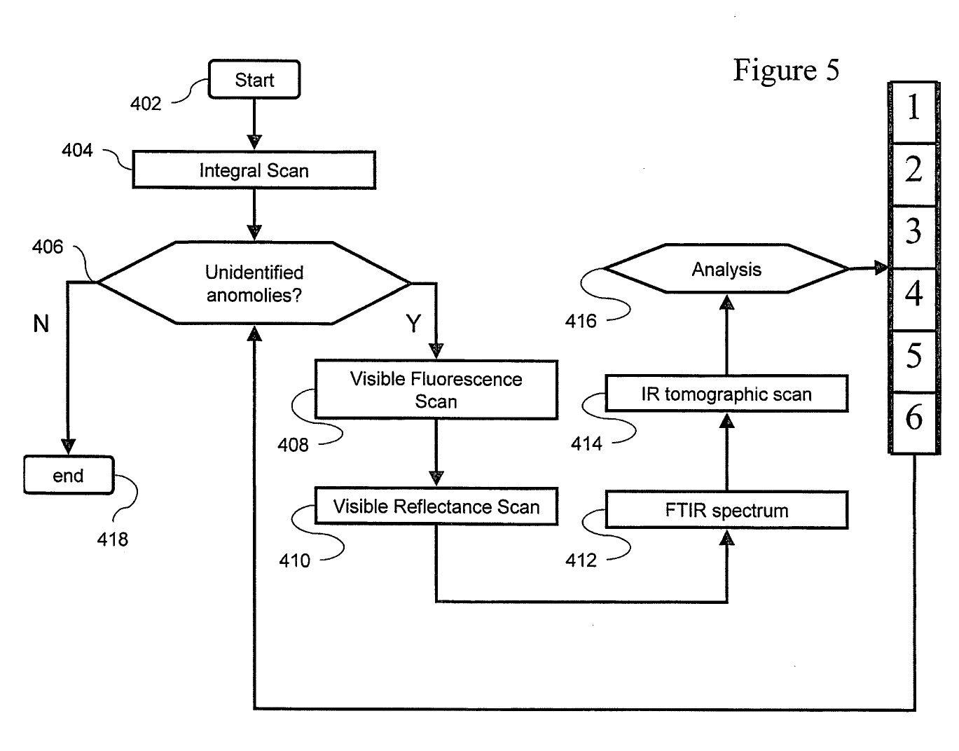

FIG. 5 is a flow chart of a method to identify a skin lesion according to the current invention. The diagnostic session starts 402 by conducting an integral scan 404 of the skin of the patient being examined to identify locations of potential lesions. Particularly, in the embodiment of FIG. 5, the integral scan is of contrast in total intensity of a wide band (from 2-10?m) of passive (black body) MIR radiation. Location of anomalies in the emitted black body MIR radiation are noted. Also the doctor notes visually, the locations of suspicious visible abnormalities in the skin (anomalies in reflected visible light). If there are any unidentified anomalies, the particular location of the anomaly is scanned in a spectral mode. First the skin is irradiated with ultraviolet light and a fluorescent spectrum is measured 408 in the visible band. Then the skin is irradiated with white light and a visible reflectance spectrum 410 is measured (note this is a wide spectrum which also includes measurements in the NIR range as above). Finally, the light source is turned off and a passive infrared spectrum of black body radiation is measured 412. Finally the area of the lesion is scanned using tomographic techniques in the IR range passively measuring black body radiation to determine the shape of the lesion both on the skin surface and at depth 414. The lesions is identified based on the results of above spectral scans and the location determined by the integral and tomographic scans by analyzing 416 as follows: 1) if the visible reflectance spectrogram has a plateau shape and the lesion has a higher heat (passive MIR) flux than normal skin and tomography shows that the increased IR flux can be identified at a depth of more than 5 mm under the skin surface, the patient is diagnosed with dangerous melanoma and sent for immediate surgery; 2) if the visible reflectance spectrogram has a plateau shape and there is high MIR flux, but tomography shows that the depth of the lesion is less than 5 mm, the patient diagnosed as having a less dangerous melanoma and is sent to have the lesion “burned” with liquid nitrogen and a deep biopsy and nodal investigation;

3) if the visible spectrum does not have a plateau shape, but has increased reflectance in the NIR range (at 900 mm) and there is increased heat flux to a depth of greater than 5 mm then the lesion is diagnosed as a dangerous cancer precursor and sent for surgical removal; 4) if the visible spectrogram does not show plateau behavior; but there is increased reflectance at 900 nm without increased heat flux at depths below 5 mm, the lesion is diagnosed as a less dangerous potential cancer precursor and the patient is put on close observation; 5) if the visible spectrogram has a positive slope, there is no elevation of NIR reflectance, but there is an increase in fluorescence over normal skin, and there is no increased heat flux, then the lesion is diagnosed as a benign Seborrheic keratosis; 6) if the visible spectrogram has a positive slope, there is no elevation of NIR reflectance, but there is an decrease in fluorescence over normal skin and there is no increased heat flux, then the lesion is diagnosed as a suspected benign compound nevus and the patient is kept under observation for possible pathologic transformations. If there are more unidentified anomalies 406 then the spectrographic 408-412, tomagraphic 414, and analysis 416 steps are repeated for each anomalous zone. If there are no more unidentified anomalous zones, then the diagnostic session is ended 418.

FIG. 6 illustrates a second embodiment of the current invention. In the embodiment of FIG. 6, the skin 20b of a patient is investigated using a probe 12b having an illumination fiber 14g connected to a light source 22b. Probe 12b also contains a pick-up fiber 16b connected to a spectrometer 502. Spectrometer 502 measures simultaneously measures radiation in multiple bands in the visible, NIR and MIR bands using a detector system 504 which may be an array of multiple detectors, each detector measuring a different frequency band. Alternatively, detector system 504 can be a interferometer producing an interference spectrum which is interpreted by a processor, which is a PC 28b by means of Fourier transform analysis. Under any conditions the measurements of detector system 504 are sent to PC 28b via interface electronics and PC 28b displays the results as a spectrogram on a monitor 30b. PC 28b also is connected to a first control cable 506a to control light source 22b to provide illumination either in the ultraviolet or the visible range in order to measure visible fluorescence or reflectance respectively (visible reflectance and fluorescence can not be measured simultaneously since the measured signal is in the same band), and a second control cable 506b to control detector system 504. In an alternative embodiment, all components (except for probe 12b) are located inside a small portable box (the processor being a dedicated processor rather than a stand alone PC 28b).

FIG. 7 shows a third embodiment of a scanner assembly 600 according to the current invention Particularly scanner assembly 600 includes an active visible sensor assembly 602, which is a bundle of five optical fibers, four illumination fibers 14h-14k and a pick up fiber 16c shown in cross section 18b. Visible light does not appreciably penetrate skin, therefore the visible sensor assembly 602 is focused by lens 610c onto a point 612 on the surface of skin 20c. Scanner assembly 600 also includes two passive MIR sensor assemblies 602604a and 604b, which are focused by lenses 610a and 610b respectively from opposite angles at a point 7 mm below point 612. Thus as scanner assembly moves along in scanning direction 606, visible sensor assembly 602 detects discoloration (or fluorescence) of the skin surface along a line, while simultaneously MIR sensor assemblies 604a and 604b measure black body MIR radiation from two directions along the same line in order to gauge the depth of a lesion 614. Thus the location of the lesion is found based both on measurements of both a visible light signal emitted from the skin due to reflection or fluorescence at the surface of skin 20c and a passive IR energy signal emitted as black body radiation in the MIR band from on and below the surface of skin 20c. Furthermore, due to the difference in focus of the various sensors, the location of the lesion on the surface of skin 20c and the depth lesion below the surface of skin 20c are determined simultaneously

It will be appreciated that the above descriptions are intended only to serve as examples, and that many other embodiments are possible within the spirit and the scope of the present invention

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A non-intrusive method for identifying a lesion in a skin of a subject, comprising the steps of:
d) finding a location of an anomaly of a radiation emitted by the skin, said anomaly caused by the lesion;
e) performing a spectral analysis including quantifying a first signal in a visual band and a second signal in an infrared band; and
f) identifying the lesion based on said location and a result of said spectral analysis.

2. The method of claim 1, wherein said step of identifying includes recognizing a cancer precursor.

3. The method of claim 2, wherein said recognizing is based on a measurement of an energy in a near infrared band.

4. The method of claim 1, wherein said radiation includes a visible light reflected from the skin.

5. The method of claim 1, wherein said radiation includes a visible light emitted by fluorescence of the skin.

6. The method of claim 1, wherein said radiation includes a black body medium infrared band energy emitted by the skin.

7. The method of claim 1, wherein said radiation includes energy in a broad frequency band including both infrared and visible frequencies.

8. The method of claim 1, wherein said radiation includes energy in the near infrared frequency band scattered by the skin.

9. The method of claim 1, wherein said radiation includes both a visible light reflected from the skin and a black body medium infrared band energy emitted by the skin.

10. The method of claim 1, wherein said step of finding includes the substeps:
(i) quantifying a first energy emitted from the skin without the lesion;
(ii) measuring a second energy emitted from said location, and
(iii) calculating a differential measure between said first energy and said second energy.

11. The method of claim 1, further including the steps:
g) classifying the lesion to a general category based on a characteristic of said anomaly, and
h) adapting said spectral analysis to differentiate between objects in said general category.

12. The method of claim 11, wherein said step of adapting includes choosing a frequency band for said spectral analysis, said frequency band being optimal to distinguish between at least two objects in said general category.

13. The method of claim 1, further including the step:
i) determining a depth of the lesion.

14. The method of claim 13, wherein said step of finding and said step of determining are performed simultaneously.

15. The method of claim 13, wherein said step of determining includes the substeps
(i) measuring an infrared energy emitted by said lesion.
(ii) computing a depth based on a result of said measuring.

16. The method of claim 1, further including the step:
d) measuring a fluorescence;
and wherein said step of identifying is further based on an outcome of said measuring.

17. The method of claim 1, wherein said second signal includes infrared energy within having wavelength between 5.5 and 7.5 micrometers.

18. The method of claim 1, wherein said step of performing a spectral analysis includes the substeps:
(iii) measuring a first energy measured in a first frequency band emitted at said location
(iv) quantifying a second energy measured in a second frequency band emitted at said location.
(v) calculating a differential measure between said first energy and said second energy.

19. The method of claim 1, wherein said second signal includes at least one emanation selected from the group consisting of a product of an interaction between an output of an external radiation source and the lesion, a heat flow from the lesion, light reflected from the lesion, and a black body radiation emitted by the lesion.

20. The method of claim 1, wherein said identifying includes classifying the lesion according to a plurality of categories, said categories including benign nevus, pathologic cancer precursor, and cancerous lesion.

21. A detector for identifying a lesion in a skin comprising:
a) a first sensor assembly sensitive to a first frequency band, said first sensor assembly configured to determine a location and a characteristic of an anomaly in a first radiation signal emitted by the skin, said anomaly being caused by the lesion;
b) a second sensor assembly configured to be sensitive to a second frequency band, and
c) a processor configured to identify the lesion based on said location, said characteristic and a contrast between an unmodified radiation signal in said second frequency band emitted by the skin and a second radiation signal measured at said location by said second sensor assembly.

22. The detector of claim 21, wherein said first sensor assembly includes an electronic sensor and said second sensor assembly includes said electronic sensor and a band pass filter.

23. The detector of claim 21, further comprising:
d) a visible light source for producing a light beam;
and wherein said first sensor assembly is configured to detect a reflection of said light beam from the skin.

24. The detector of claim 21, further comprising:
e) A ultra-violet light source configured to induce fluorescence of the skin;
And wherein said second sensor is configured to detect said fluorescence.

25. The detector of claim 21, wherein said processor includes at least one processing unit selected from the group consisting of a human operator, a dedicated electronic processor, and a personal computer.
